(19) United States
(12) Patent Application Publication
(10) Pub. No.: US 2014/0025145 A1
(43) Pub. Date: Jan. 23, 2014

(54) SEGMENTED ICE WRAP

(71) Applicant: Christopher Kirkman, San Diego, CA (US)

(72) Inventor: Christopher Kirkman, San Diego, CA (US)

(21) Appl. No.: 13/553,507

(22) Filed: Jul. 19, 2012

(51) Int. Cl. A61F 7/10

(52) U.S. Cl. CPC A61F 7/103
USPC 607/112; 156/292

(57) ABSTRACT

A device for transferring heat from a body part includes a segmented pouch formed from upper and lower layers of plastic that are heat bonded together at their peripheries. A fluid port is established for the pouch to allow a fluid such as water to be introduced into the pouch. Within the pouch, a plurality of substantially coplanar pockets for holding a respective plurality of frozen fluid lumps are formed by selectively heat bonding the layers together. With this arrangement, the pouch and frozen fluid lumps establish an ice wrap that is sufficiently flexible to conform to a surface of the body. An elongated strap made of nonwoven fabric is attached to the pouch and a piece of hook fastener is affixed to the strap for engagement with the nonwoven fabric to secure the device when the strap is wrapped around the pouch and body part.

### FIELD OF THE INVENTION

The present invention pertains generally to ice wraps. More particularly, the present invention pertains to reusable flexible ice wraps that can be filled and frozen by the user. The present invention is particularly, but not exclusively, useful as an ice wrap having an integral strap for attaching a flexible, frozen portion of the ice wrap to a user's body.

### BACKGROUND OF THE INVENTION

The application of ice or another cold substance to a body part is often prescribed after an injury to reduce pain and/or swelling. In addition, cold therapy can be useful to reduce pain and/or swelling in a previously injured joint or muscle, for example, after an activity such as working or exercise.

To be effective, it can be important that the cold applicator conform to the particular anatomical region requiring therapy. In this regard, the anatomical regions that often require therapy vary considerably in shape and size. These can include, for example, the knee, ankle, shoulder, elbow and wrist. In addition to the anatomical variation within a particular individual, the size and shape of different individuals varies greatly across the general population. For this reason, it is often desirable to have a cold applicator that is flexible to fit most if not all of the different anatomical shapes and sizes.

Once applied, it is generally desirable to leave the cold wrap in place for an extended period of time. Moreover, the therapeutic value of a cold application can often be increased if the application is accompanied by a moderate amount of compression. In this regard, it is often desirable to affix the cold applicator to the affected body part to relieve the person from the chore of holding the cold applicator in place. For this purpose, various attachment methods have been suggested. In most cases, it is desirable that the cold applicator be able to be applied and affixed by the person requiring treatment, and some instances can be accomplished with only the use of one hand. Further, it is desirable that the cold applicator be reusable.

In light of the above, it is an object of the present invention to provide a reusable, flexible ice applicator that is conformable to several different parts of the body. Still another object of the present invention is to provide an ice applicator system that can be easily affixed to the user. Yet another object of the present invention is to provide a Segmented Ice Wrap and corresponding methods of use which are easy to use, relatively simple to implement, and comparatively cost effective.

### SUMMARY OF THE INVENTION

In accordance with the present invention, a device for transferring heat from a body part includes a segmented pouch. To create the pouch, two flexible plastic layers (i.e. an upper and lower layer) are heat bonded together at their peripheries. In addition, a fluid port is established for the pouch to allow a fluid such as water to be introduced into the pouch. Within the pouch, a plurality of substantially coplanar pockets for holding a respective plurality of frozen fluid lumps are formed by heat bonding the layers together in a preselected pattern. With this arrangement, the pouch and frozen fluid lumps establish an ice wrap that is sufficiently flexible to conform to a surface of the body such as the knee, ankle, wrist, etc.

For the device of the present invention, an elongated strap is attached to the pouch to secure the pouch in place once the pouch and frozen fluid lumps have been applied to the surface of the body. Structurally, for the present invention, the strap can be made of nonwoven fabric and it is sized to be long enough to wrap around the pouch and a targeted body part.

In one embodiment, a first end of the strap is attached to the pouch and a piece of hook fastener is affixed to the strap adjacent the first end. With this arrangement, the hook fastener and nonwoven fabric establish a fastening system when the strap is folded over and across the pouch to maintain the pouch against the surface of the body part.

In another embodiment, a first end of the strap is attached to the pouch and a piece of hook fastener is affixed to the strap adjacent a second end of the strap. With this arrangement, the hook fastener attaches to the nonwoven fabric after the strap has been fully wrapped around the pouch and body to hold the pouch in place against the body and provide compression.

Alternatively, a first piece of hook fastener can be affixed to the strap adjacent to the first end of the strap (on a first side of the strap) and a second piece of hook fastener can be affixed to the strap adjacent the second end of the strap (on a second side of the strap). With this arrangement, the pouch can be applied to a targeted body part and the strap wrapped around the body part and over the pouch. A first fastening attachment is then made between the strap and the first piece of hook fastener once the strap covers the pouch. The remainder of the strap is then wrapped around the body part and a second fastening attachment is made between the strap and the second piece of hook fastener to secure the strap in place.

For the present invention, the fluid port for introducing fluid into the pouch can include a tapered input channel that is formed at a peripheral section of the pouch by heat bonding the upper and lower layers together in a preselected pattern. In one embodiment, a one-way fluid valve such as a duck-bill valve may be inserted into the input channel and attached to the upper and lower layers. Alternatiely, a sliderless plastic zipper may be formed at a peripheral section of the pouch.

To establish the plurality of coplanar pockets for holding a respective plurality of frozen fluid lumps, the upper layer and the lower layer are connected in a pattern that is formed by heat bonding the layers together. The pattern can include a first plurality of partitions with each partition in the first plurality formed with fluid channels that interconnect adjacent pockets. For the pattern, each partition in the first plurality is aligned substantially parallel to other partitions in the first plurality. Also, the pattern includes a second plurality of partitions with each partition in the second plurality formed with fluid channels that interconnect adjacent pockets. For the pattern, each partition in the second plurality is aligned substantially parallel to other partitions in the second plurality and substantially perpendicular to the partitions of the first plurality.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a segmented ice wrap device in accordance with the present invention shown after applying the pouch portion of the device on a person's knee;

FIG. 2 is a plan view of the segmented ice wrap device shown in FIG. 1;

FIG. 2A is a cross sectional view of the pouch portion of the segmented ice wrap shown in FIG. 2 as seen along line 2A-2A in FIG. 2;

FIG. 3 is a detail view of a portion of a device enclosed by line 3-3 in FIG. 2 illustrating a first pattern of heat bond dots for creating a plurality of coplanar pockets for holding a respective plurality of frozen fluid lumps;

FIG. 4 is a detail view of a portion of a device enclosed by line 3-3 in FIG. 2 illustrating another pattern of heat bond dots for creating a plurality of coplanar pockets for holding a respective plurality of frozen fluid lumps;

FIG. 5 shows another embodiment of a device in accordance with the present invention having a tapered fluid port; and

FIG. 6 shows another embodiment of a device in accordance with the present invention having a fluid port formed with a constriction.

### DESCRIPTION OF THE PREFERRED EMBODIMENTS

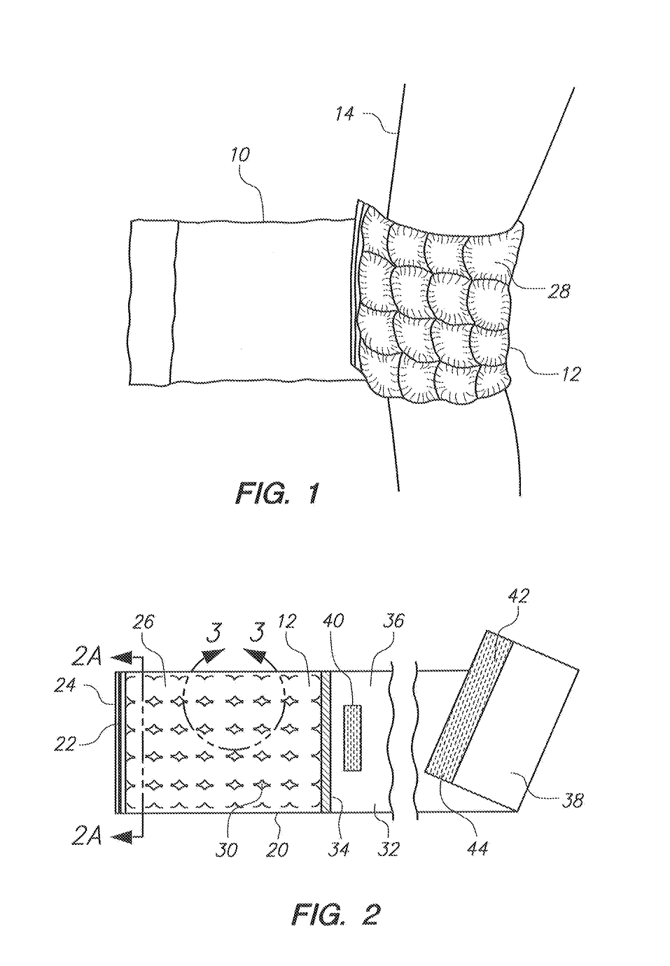

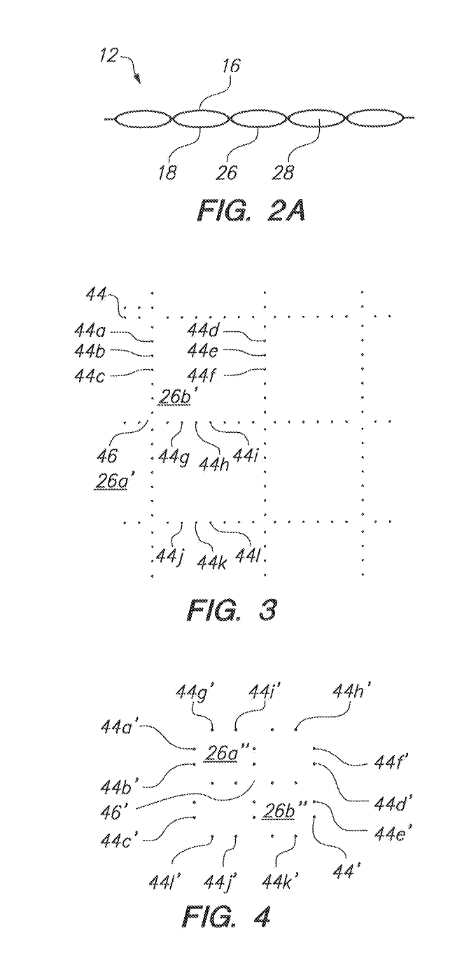

Referring initially to FIG. 1, an ice wrap device 10 having a segmented pouch 12 in accordance with the present invention is shown. Specifically, FIG. 1 shows the device 10 after the pouch 12 has been applied to a surface of a person's leg 14 to cover their knee. As best appreciated by cross referencing FIGS. 1, 2 and 2A, the pouch 12 may be generally rectangular shaped and may be formed from an upper layer 16 and a lower layer 18 of plastic that are heat bonded together at a peripheral section 20 to create the pouch 12.

FIG. 2 shows that a fluid port 22 can be established for the pouch 12 at a peripheral section 24 to allow a fluid such as water or gel to be introduced into the pouch 12. For the embodiment shown in FIG. 2, the fluid port 22 can be a sliderless plastic zipper sometimes referred to generically as a so-called Ziploc closure.

Continuing with cross-reference to FIGS. 1, 2 and 2A, it can be seen that within the pouch 12, a plurality of substantially coplanar pockets 26 for holding a respective plurality of frozen fluid lumps 28 are formed by heat bonding the layers 16, 18 together in a preselected pattern 30. With this arrangement, the pouch 12 and frozen fluid lumps 28 establish an ice wrap device 10 that is sufficiently flexible to conform to a surface of the body such as the knee, ankle, wrist, etc. For example, the plastic sheets used for the upper layer 16 and lower layer 18 may be made of a clear and/or transparent material that is sufficiently stretchable to accommodate a freezing of the fluid into lumps 28 within the respective pockets 26. For example, the sheet material may be a combination of nylon and polyethylene having a thickness of less than approximately four mils.

FIGS. 1 and 2 also show that the device 10 includes an elongated strap 32 having an end 34 that is attached to the pouch 12. For example, the pouch and strap 32 may be attached together using heat bonding or adhesive bonding. For the device 10, the strap 32 can be made of nonwoven fabric and is typically sized to be long enough to wrap around the pouch and a targeted body part such as the knee, ankle, elbow, etc. For example, the nonwoven fabric may be made of polypropylene and can include a texture. With this arrangement, the strap 32 can be used to secure the pouch 12 in place once the pouch 12 and frozen fluid lumps 28 have been applied to the surface of the body (see FIG. 1).

As best seen in FIG. 2, the strap 32 includes a side 36 and an opposed side 38. FIG. 2 also shows that a piece of hook fastener 40 can be affixed to the nonwoven fabric of the strap 32 on the side 36 of the strap 32 and adjacent the end 34 of the strap 32. Continuing with FIG. 2, it can be seen that another piece of hook fastener 42 can be affixed to the nonwoven fabric of the strap 32 on the side 38 of the strap 32 and adjacent an end 34 (opposite end 34) of the strap 32. For example, the hook fastener 40, 42 can be adhesively bonded and/or sewn on to the nonwoven fabric of the strap 32.

With this arrangement shown in FIGS. 1 and 2, the pouch 12 can be applied to a targeted body part and the strap 32 wrapped around the body part and over the pouch 12. A first fastening attachment is then made between the nonwoven fabric of the strap 32 and the piece of hook fastener 40 once the strap 32 covers the pouch 12. The remainder of the strap 32 is then wrapped around the body part and a second fastening attachment is made between the nonwoven fabric of the strap 32 and the piece of hook fastener 42 to secure the strap 32 in place.

FIGS. 3 and 4 show respective heat bond patterns 30′, 30″ for establish the plurality of coplanar pockets 26′; 26″ for holding a respective plurality of frozen fluid lumps (see FIG. 2A). The patterns 30′, 30″ shown can be repeated throughout the pouch 12 as shown in FIG. 2. Pattern 30′ shown in FIG. 3 is designed to produce relatively large ice lumps that are slightly rectangular and have a large side dimension of about one inch (25.4 mm). On the other hand, pattern 30″, shown in FIG. 4, is designed to produce relatively small ice lumps that are substantially square and have a side dimension of about one half inch (12.7 mm).

As shown in FIG. 3, the layers 16, 18 (see FIG. 2A) are connected in a pattern of heat bond dots 44 that are formed by applying heat to the layers 16, 18 at the location of each dot 44. Also shown in FIG. 3, the pattern 30′ can include a plurality of dots 44a-c that are arranged linearly along a first line that establishes a first partition. It can also be seen that the pattern 30′ can include a plurality of dots 44d-f that are arranged linearly along a second line that establishes a second partition. Thus, the pattern 30′ establishes a first plurality of partitions in which each partition in the first plurality is aligned substantially parallel to other partitions in the first plurality.

Also shown in FIG. 3, the pattern 30′ can include a plurality of dots 44g-i that are arranged linearly along a third line that establishes a third partition. It can also be seen that the pattern 30′ can include a plurality of dots 44j-l that are arranged linearly along a fourth line that establishes a fourth partition. Thus, pattern 30′ establishes a second plurality of partitions in which each partition in the second plurality is aligned substantially parallel to other partitions in the second plurality. In addition, as shown, for the pattern 30′, each partition in the second plurality is aligned to be substantially perpendicular to the partitions of the first plurality. With this pattern 30′ of dots 44, as illustrated by FIG. 3, a plurality of fluid channels 46 are established that interconnect adjacent pockets 26a′, 26b′.

As shown in FIG. 4, the layers 16, 18 (see FIG. 2A) are connected in a pattern of heat bond dots 44′ that are formed by applying heat to the layers 16, 18 at the location of each dot 44′. Also shown in FIG. 4, the pattern 30″ can include a plurality of dots 44a′-c′ that are arranged linearly along a first line that establishes a first partition. It can also be seen that the pattern 30″ can include a plurality of dots 44d′-f′ that are arranged linearly along a second line that establishes a second partition. Thus, the pattern 30″ establishes a first plurality of partitions in which each partition in the first plurality is aligned substantially parallel to other partitions in the first plurality.

Also shown in FIG. 4, the pattern 30″ can include a plurality of dots 44g′-i′ that are arranged linearly along a third line that establishes a third partition. It can also be seen that the pattern 30″ can include a plurality of dots 44j′-l′ that are arranged linearly along a fourth line that establishes a fourth partition. Thus, pattern 30″ establishes a second plurality of partitions in which each partition in the second plurality is aligned substantially parallel to other partitions in the second plurality. In addition, as shown, for the pattern 30″, each partition in the second plurality is aligned to be substantially perpendicular to the partitions of the first plurality. With this pattern 30″ of dots 44′, as illustrated by FIG. 4, a plurality of fluid channels 46′ are established that interconnect adjacent pockets 26a″, 26b″. Although FIGS. 3 and 4 show two exemplary patterns, it is to be appreciated that the number of dots per pocket can be varied, that linear heat bond segments may be used in place of a linear array of dots, that the pockets may be oval, square, rectangular or some other shape, and that the partitions as defined above may be non-perpendicular.

FIG. 5 shows an example of a fluid port 22′ for use in the device 10 to introduce a fluid such as water into the pouch 12′. As shown, the fluid port 22′ can include a tapered input channel 48 that is formed at a peripheral section 24′ of the pouch 12′ by heat bonding layers 16, 18 (see FIG. 2A) together in the pattern shown (heat bond shown in crossed hatching). Perforations 50a,b on each side on the input channel 48 can be provided allowing a user to tear the perforations 50a,b to establish tabs which can then the tied to close the fluid port 22′ after filling. An optional, a one-way fluid valve 52 such as a duck-bill valve may be inserted into the input channel 48 and attached to the upper and lower layers 16, 18 (see FIG. 2A), as shown.

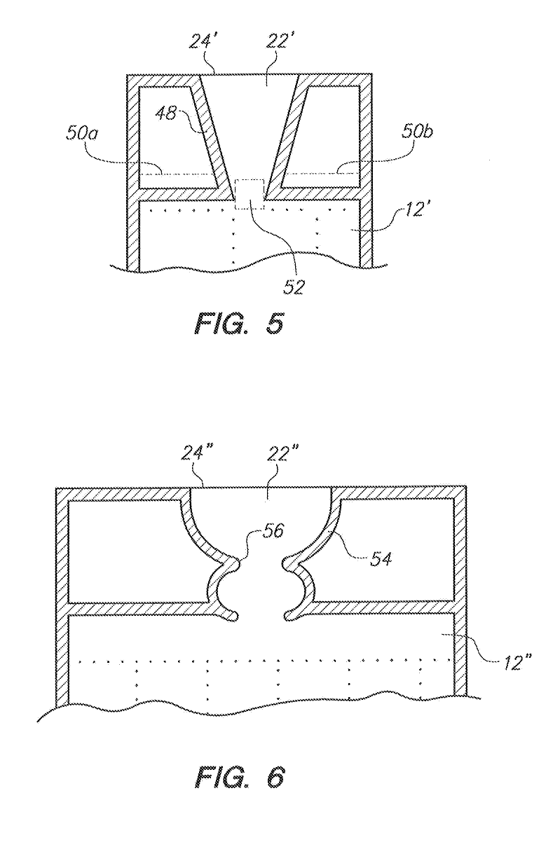

FIG. 6 shows another example of a fluid port 22″ for use in the device 10 to introduce a fluid such as water into the pouch 12″. As shown, the fluid port 22″ can include an input channel 54 that is formed at a peripheral section 24″ of the pouch 12″ by heat bonding layers 16, 18 (see FIG. 2A) together in the pattern shown (heat bond shown in crossed hatching). As shown, input channel 54 is formed with constriction 56 which provides a venture effect to cause the input channel 54 to close at the constriction 56 in response to a flow of fluid out of the input channel 54 from the pouch 12″.

While the particular segment ice wrap and corresponding methods of manufacture as herein shown and disclosed in detail are fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A device for transferring heat from a body part which comprises:
a segmented pouch having a periphery, the pouch being formed with a plurality of substantially coplanar pockets for holding a respective plurality of frozen fluid lumps, wherein the pouch is flexible to conform the pouch against a surface of the body part;
an elongated strap having a first end and a second end, and having a top side and a bottom side, wherein the first end of the strap is affixed to the periphery of the pouch; and
a fastener attached to the top side of the strap and adjacent the first end of the strap wherein, with the segmented pouch positioned against the surface of the body part, the strap is folded over and across the pouch to secure the bottom side of the strap against the fastener to maintain the pouch against the surface of the body part for heat transfer therebetween.

2. A device as recited in claim 1 wherein the segmented pouch comprises:
an upper layer having a periphery, wherein the periphery of the upper layer has a first section and a second section;
a lower layer having a periphery dimensioned and sectioned substantially the same as the upper layer, wherein the respective first peripheral sections of the upper layer and the lower layer are permanently joined together to establish a fluid tight seal along a first section periphery of the pouch, and wherein the respective second peripheral sections are selectively joined together to create a temporary fluid tight seal along a second section periphery of the pouch;
a first plurality of partitions connecting the upper layer to the lower layer within the peripheries of the upper and lower layers, wherein each partition in the first plurality is formed with fluid channels interconnecting adjacent pockets, and each partition is aligned substantially parallel to other partitions in the first plurality; and
a second plurality of partitions connecting the upper layer to the lower layer within the peripheries of the upper and lower layers, wherein each partition in the second plurality is formed with fluid channels interconnecting adjacent pockets, and each partition in the second plurality is aligned substantially parallel to other partitions in the second plurality, and further wherein the partitions of the second plurality are substantially perpendicular to the partitions of the first plurality to establish the plurality of coplanar pockets.

3. A device as recited in claim 2 wherein a fluid port is formed along the second section periphery of the pouch within the periphery of the pouch.

4. A device as recited in claim 2 wherein the pouch is rectangular.

5. A device as recited in claim 2 wherein the first layer and the second layer are made of a combination of nylon and polyethylene and are sufficiently stretchable to accommodate a freezing of the fluid into lumps within the respective pockets.

6. A device as recited in claim 5 wherein the frozen fluid is ice.

7. A device as recited in claim 5 wherein the frozen fluid lumps are each defined by coplanar sides, and wherein each side of each frozen fluid lump is less than approximately one inch.

8. A device as recited in claim 1 wherein the strap is stretchable and is made of a nonwoven material.

9. A device as recited in claim 8 wherein the nonwoven material is polypropylene.

10. A device as recited in claim 1 wherein the fastener is a first fastener and the device further comprises a second fastener attached to the bottom side of the strap adjacent the second end of the strap for securing the second fastener against the top side of the strap to maintain the pouch against the surface of the body part for heat transfer therebetween.

11. A device as recited in claim 10 wherein the first fastener and the second fastener respectively include a plurality of hook-like structures, and wherein the hook-like structures establish a secure engagement with the strap when the fastener is placed in contact with the nonwoven material of the strap.

12. A device for transferring heat from a body part which comprises:
a segmented pouch having a periphery, the pouch being formed with a plurality of substantially coplanar pockets for holding a respective plurality of frozen fluid lumps, wherein the pouch is flexible to conform the pouch against a surface of the body part;
an elongated strap affixed to the periphery of the pouch and made of a nonwoven fabric; and
a hook fastener affixed to the strap and engageable with the nonwoven fabric fasten the hook fastener with the nonwoven fabric, the hook fastener attached to the strap at a position to engage the nonwoven fabric when the strap is folded over and across the pouch to secure the pouch against the surface of the body part for heat transfer therebetween.

13. A device as recited in claim 12 wherein the elongated strap has a first end attached to the pouch and a second end and the hook fastener is affixed to the strap adjacent the first end.

14. A device as recited in claim 12 wherein the elongated strap has a first end attached to the pouch and a second end and the hook fastener is affixed to the strap adjacent the second end.

15. A device as recited in claim 12 wherein the elongated strap has a first end attached to the pouch and a second end and wherein a first hook fastener portion is affixed to the strap adjacent the first end and a second hook fastener portion is affixed to the strap adjacent the second end of the strap.

16. A method for manufacturing a device for transferring heat from a body part which comprises the steps of:
heat bonding a first peripheral section of a lower layer with a first peripheral section of an upper layer to establish a pouch;
heat bonding portions of the lower layer to portions of the upper layer to create partitions to establish plurality of substantially coplanar pockets for holding a respective plurality of frozen fluid lumps, wherein the pouch is flexible to conform the pouch against a surface of the body part;
attaching an elongated strap made of a nonwoven fabric to the pouch;
attaching a hook fastener to the strap.

17. A method as recited in claim 16 further comprising the step of heat bonding a second peripheral section of the lower layer with a second peripheral section of the upper layer to establish a fluid port for the pouch.

18. A method as recited in claim 16 wherein the step of heat bonding portions of the lower layer to portions of the upper layer to create partitions to establish plurality of substantially coplanar pockets comprises the steps of:
creating a first plurality of partitions connecting the upper layer to the lower layer within the peripheries of the upper and lower layers, wherein each partition in the first plurality is formed with fluid channels interconnecting adjacent pockets, and each partition is aligned substantially parallel to other partitions in the first plurality; and
creating a second plurality of partitions connecting the upper layer to the lower layer within the peripheries of the upper and lower layers, wherein each partition in the second plurality is formed with fluid channels interconnecting adjacent pockets, and each partition in the second plurality is aligned substantially parallel to other partitions in the second plurality, and further wherein the partitions of the second plurality are substantially perpendicular to the partitions of the first plurality to establish the plurality of coplanar pockets.

19. A method as recited in claim 16 further comprising the step of affixing a duck bill valve to a second peripheral section of the lower layer and a second peripheral section of the upper layer.

20. A method as recited in claim 16 further comprising the step of attaching a sliderless plastic zipper to a second peripheral section of the lower layer and a second peripheral section of the upper layer.
